(19) United States
(12) Patent Application Publication
(10) Pub. No.: US 2022/0138523 A1
(43) Pub. Date: May 5, 2022

(54) METHOD FOR HUMAN IMMORTALITY

(71) Applicant: Ning Li, Houston, TX (US)

(72) Inventor: Ning Li, Houston, TX (US)

(21) Appl. No.: 17/134,318

(22) Filed: Dec. 26, 2020

Related U.S. Application Data
(60) Provisional application No. 63/108,567, filed on Nov. 2, 2020.

(51) Int. Cl. G06N 3/00; G16H 40/63

(52) U.S. Cl. CPC G06N 3/008; G16H 40/63

(57) ABSTRACT

A method of achieving human immortality is disclosed. The disclosed method includes implanting a human brain microchip into a person's brain, capturing personal memory data from that person's brain to the brain microchip, retrieving personal memory data from the human brain memory microchip, integrating the memory data with a robot microchip to form a human-machine microchip, implant the human-machine microchip into a selected human-machine body to form a new human and testing the new human for normal functionalities.

### CROSS-REFERENCE TO RELATED APPLICATIONS

This is a non-provisional of U.S. Provisional Application Ser. No. 63/108,567, filed on Nov. 2, 2020, and entitled “System and Method for Perpetual Human Life,” which is incorporated herein by reference in its entirety and to which priority is claimed.

### FIELD OF THE INVENTION

The subject matter of the present disclosure relates generally to a method of preserving life and memory of human beings to achieve human immortality.

### BACKGROUND OF THE INVENTION

Human longevity and immortality are the greatest fantasy and the most beautiful ideal and hope in human history. Qin Shi Huang, the first Emperor of China more than 2,000 years ago, sent 3,000 boys and girls to Japan to find herbs that can make people live longer. But after more than 2,000 years of history, the best of mankind has not been realized. Only now, with the rapid development of modem technology, provides such opportunities and windows for human immortality. Recent developments have shown that it's possible to implant microchips in a brain. These microchips would be used to treat brain injuries and diseases that directly affect brain functions such as Alzheimer's. These new technologic advances make it possible to find a method to preserve human memory and achieve human immortality.

It is, therefore, desirable for a method to provide mechanism for human immortality.

### OBJECTIVES AND SUMMARY OF THE INVENTION

The main objective of the present disclosure is to preserve personal memory data for a person and transfer that personal memory data to a robotic body to achieve immortality.

In accordance with some embodiments, an intelligent immortal human brain microchip is fabricated and implanted into a human brain to read personal memory data of that person, after leaving the brain microchip in the brain for a period of time, the human brain microchip is removed from the brain, personal memory data stored on the human brain microchip is then retrieved and inspected, the memory data may be modified if necessary, the memory data is then integrated with a robot microchip to form a man-machine microchip, then select a desired man-machine body for the person and implant the man-machine microchip in the selected man-machine body, a final inspection is conducted on the man-machine body to make sure it functions normally like a human being. This creates a new human with the memory of the original person.

### BRIEF DESCRIPTION OF THE DRAWINGS

The present disclosure is further described in terms of exemplary embodiments. These exemplary embodiments are described in detail with reference to the drawings. These embodiments are non-limiting exemplary embodiments, in which like reference numerals represent similar structures throughout the several views of the drawings, and wherein:

FIG. 1 illustrates, in flowchart form, an example of one embodiment of a technique for preserving human memory to achieve human immortality;

FIG. 2 illustrates a human brain implanted with immortal human brain microchip according to one embodiment;

FIG. 3 illustrates, in flowchart form, an example of one embodiment of a technique for retrieving personal memory data from a human brain microchip and inspecting memory data;

FIG. 4 illustrates, in flowchart form, an example of one embodiment of a technique for integrating memory data with a robot microchip.

### DETAILED DESCRIPTION OF THE EMBODIMENTS

The following description is presented to enable any person skilled in the art to make and use the present disclosure and is provided in the context of a particular application and its requirements. Various modifications to the disclosed embodiments will be readily apparent to those skilled in the art, and the general principles defined herein may be applied to other embodiments and applications without departing from the spirit and scope of the present disclosure. Thus, the present disclosure is not limited to the embodiments shown, but is to be accorded the widest scope consistent with the claims.

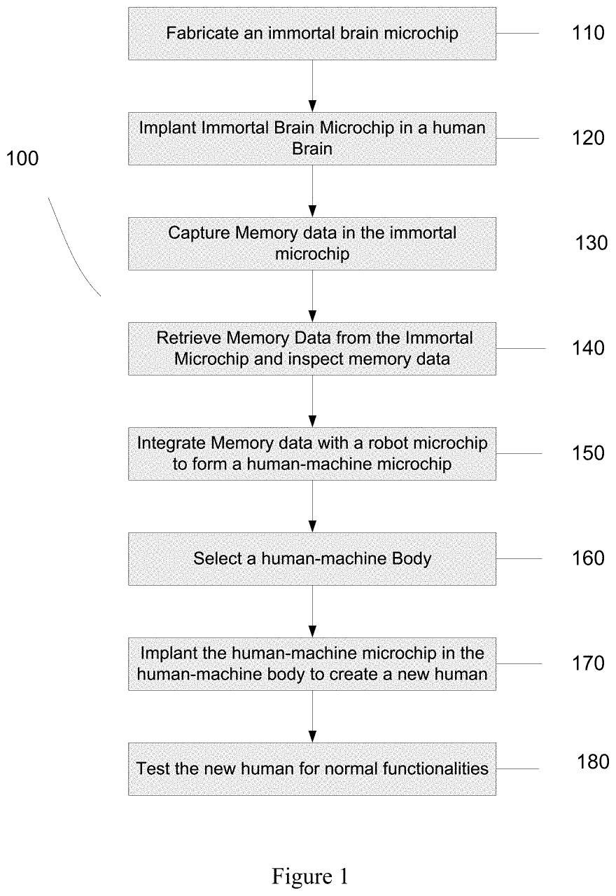

Referring now to FIG. 1, a method 100 for preserving human memory to achieve human immortality is illustrated.

### Fabricate Intelligent Microchips for Human Immortality

The immortal human brain microchip used to capture human memory in this disclosure is a learning-type high-intelligence capturing chip. The immortal human brain microchip has smart mesh fiber contacts, which can capture a wealth of personal information such as personal consciousness and memory, emotion, and intelligence.

Using nanotechnology, the aggregation of nanoparticles in the brain can be measured. The microchips may be implanted in different regions of the brain for correct recording and collection of the personal information. The microchip may have over 3000 channels and supports remote data wireless transmission. The energy consumption of the microchip is low and does not need to be equipped with any battery, so the microchip may be directly implanted into the human scalp for information capturing. In some embodiments, the microchip may be wirelessly charged.

In the latest sci-fi movie “The Matrix”, there was a plot of brain-computer transmission, controlling equipment and even changing reality. This movie provides us a beautiful imagination of making human immortal microchips.

In the eighteenth century, human understanding of the brain can be summarized in four aspects. 1. Damage to the brain stem will interfere with sensory, motor and thinking functions, and even lead to death. 2) The brain communicates with other parts of the body through nerves. 3) The brain is composed of parts with different characteristics and they have different functions. 4) The brain works like a machine and follows the laws of nature.

The breakthrough of human knowledge about the brain in the 19th century is reflected in four aspects: 1) nerves are wires instead of water pipes, 2) different functions are located in different parts of the brain, 3) the nervous system is the product of evolution, 4) the neuron is the brain's basic structure and functional unit. In 1873, the Italian biologist Camillo Golgi's discovery of neuron unveils an important secret in the brain.

The cerebral cortex is the center hub of the nerve system. It is the foundation of all high-level mental activities, including intelligence and memory. Research has found that the area of human cerebral cortex is about 1.5 m2, as big as the size of four daily newspaper pages, whereas the area of chimpanzee cerebral cortex is about the size of one newspaper page, the area of monkey cerebral cortex is as big as a postcard, and mice's cerebral cortex is as big as a stamp.

We now know that cranial nerves are independent entities that communicate with each other. Neurons works through neuronal action potential, which can be conducted along the nerve cell membrane.

The connection of neurons is done through synapses. Nerve impulses complete the overall process of synaptic transmission across neurons, and most of the transmission of nerve synapses requires the help of a substance called neurotransmitter. How are nerve impulses completed through synapses? The existence of synapses between brain neurons is the realization of information transmission and constitutes the basis of thinking and memory activities.

The functions of the cerebral hemispheres have been highly differentiated after human adulthood. The functions of one hemisphere will not be transferred to the other side, and scientists have found that the cognitive function of the right hemisphere is advantageous and dominant. Many advanced imagery thinking, observation and comprehensive abilities are concentrated on the right hemisphere.

The British physiologist Richard Caton laid the foundation of electroencephalogram (EEG) in 1875. He used a voltage-sensitive device to record electrical activity on the brain surfaces of dogs and rabbits, and then conducted electrical current measurement on the human brain. This is the beginning of EEG. More importantly, it shows that the basic activities of the human brain can be recorded.

The brain's process of remembering billions is the role of hippocampus, amygdala, cerebellum and cerebral cortex. What is memory? The best answer currently given by neuroscientists is that a memory is a way of linking a group of preserved brain neurons. Activities in the hippocampus and nearby areas convert short-term memories into long-term memories, and the neural connections formed when the cortex is stimulated are strengthened and linked to the emotional state at the time of formation.

The hippocampus is reinforced with memory, but it is not responsible for preservation. Once the memory is encoded, it stays in the cortex area where the information was originally perceived and processed. When needed or when emotions have been sent, this memory is then activated as working memory to put into use, so our optical fiber probes need to be placed in different cerebral cortex areas for a period of one to six months, so that different memories of different people can be detected and collected. The longer the time, the more memory information can be captured.

We can also use a method of large number of stimulus to ensure that a large amount of information is obtained in a short time. For instance, we ask lots of questions, such as, what is your name, what are names of your parents, brothers and sisters, what are the major events you have experienced, what are the key things you deal with, etc., to stimulate the occurrence of his memories, so that we can record more in a shorter time frame.

Emotional memory, the neural link caused by events with strong emotions, uses the amygdala as the hub, which can ensure that the body quickly responds to a crisis. The existence of habit and skill memories is unconscious, and these memories depend on the basal ganglia and cerebellum.

The structure related to memory is the amygdala, which functions similarly to the hippocampus. In addition, certain parts of the diencephalon located in the prefrontal cortex are also involved in the memory process.

Human memory is divided into declarative memory and non-declarative memory. Declarative memory is the memory of past experiences and events. Non-declarative memory includes many types, such as skills, habitual emotional memory, motor conditioning, etc. Usually declarative memory is conscious memory. The formation of non-declarative memory requires long repetitive practice, but it cannot be forgotten, such as swimming, driving, playing ball, etc.

The memory storage part of the brain is fixed and clear. The brain has multiple memory systems, and different types of memories are formed and stored in different areas of the cortex. Declarative and habitual memories are formed in the motor cortex and basal ganglia. Emotional memory depends on the amygdala, and motor conditioning requires the cerebellar cortex and its deep nuclei.

Memory is permanently stored and transferable. Scientists did specific experiments. It is to punish a mouse who originally liked the dark to form a conditioned reflex to avoid the dark. As a result, scientists extracted a kind of fear hormone from the brain tissue of that mouse. Injection of this fear hormone into untrained mice can also produce the same conditioned reflex to avoid darkness. This shows that memories are stored and can be transferred to different individual bodies.

In summary, a person's memory can be extracted and transferred. Our human immortality plan is to have a high-intelligence microchip and a mesh optical fiber probe, which are implanted in the locations where memory occur and store, so as to extract effective long-term memories and short-term memories. These memories are then transferred to the brains of high-intelligence robots, thereby transferring personal memories and consciousness to achieve the great goal of human immortality.

Life and death are the iron laws of mankind for thousands of years, and they are absolutely unchangeable. The rapid development of science and technology can break this eternal law. Human beings can truly move towards the glorious stage of human history of immortality, and what remains is how to build a peaceful society where new humans and old humans live in harmony.

The first step 110 in achieving human immortality is to fabricate immortal microchips to be implanted into human brain to capture memory information stored in the human brain using modern technology.

Implant Human Immortal Microchip into a Human Brain

The part of the brain that produces personal consciousness is the basal nucleus, cerebellum, hypothalamus, amygdala, etc. in conjunction with the cerebral cortex. We can use the method of minimally invasive surgery to place the immortal microchip and optical fiber mesh contacts in the different parts of the above-mentioned brain to obtain the wealth of personal information and data, including obtaining his/her name, personal experience and memory, his/her unique intelligence and personality and emotional characteristics.

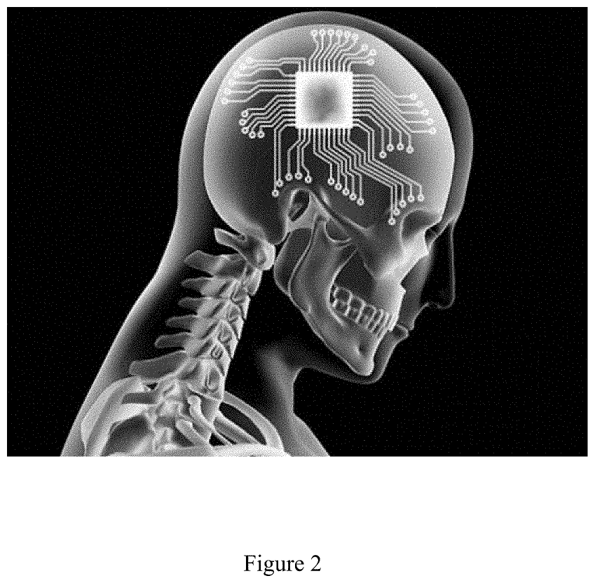

The second step 120 in achieving human immortality is to implant an immortal microchip into a human brain to capture memory information stored in the human brain. The size of such microchip may be close to 20 mm in diameter and 6 mm in width according to one embodiment. This immortal human brain microchip is placed in the brain for 30-180 days to obtain as much brain information as possible, and then the microchip is taken out for inspection and adjustment. FIG. 2 illustrates a human brain with immortal microchips implanted.

Step 130 is to capture memory data of the person and store the memory data in the immortal human brain microchip.

### Immortal Microchip Inspection and Modifications

Step 140 is to retrieve personal memory data from the brain microchip, inspect the memory data.

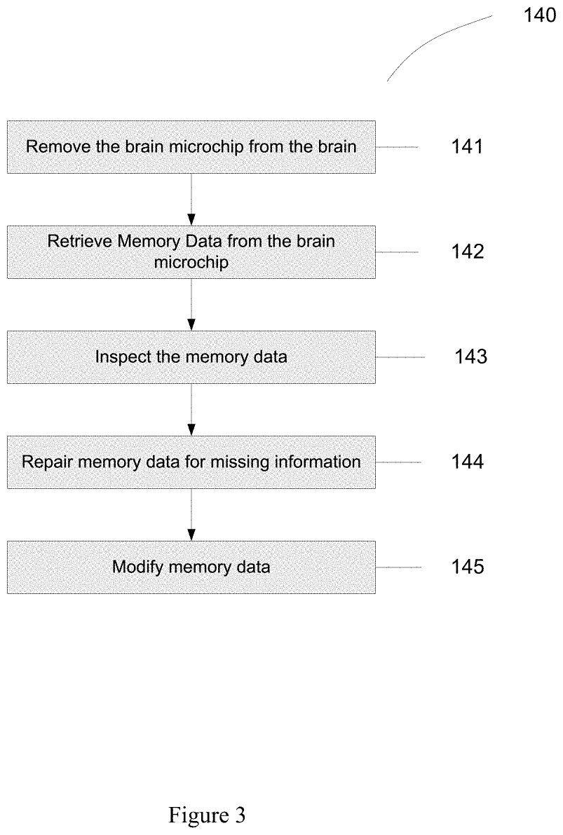

FIG. 3 illustrates detailed steps of step 140 according to one embodiment. After the brain microchip is taken out from the brain as shown in 141, personal memory data from the brain microchip is retrieved, shown as 142. A detailed inspection must be performed on the retrieved memory data, shown as 143, any missing part of the memory data must be supplemented and repaired to restore it to a normal state, shown as 144. Next, check the details of the retrieved memory data, including the privacy of the individual, and then modify the retrieved memory data according to the standards of public morals, so that memory data is healthier and more complete, as shown in 145.

Brain signals have the highest level of personal privacy. When the immortal human brain microchip reads personal desire privacy; it could mean that we have lost the final privacy, but the privacy part can be deleted during the memory data inspection stage. It is very important to protect the safety of various information in the brain, and ensure that it will not be exposed to violent criminal groups and individuals.

Obviously, this immortal microchip technology can disrupt people's sense of self-identity and initiative. Therefore, science should have boundaries. The boundaries of science are human relations and laws. Science beyond the boundaries is not allowed to exist, so the memory data retrieved from the immortal microchip taken out from a human brain must be carefully inspected and modified.

Integration of Human Memory Data with an Intelligent Robot Microchip

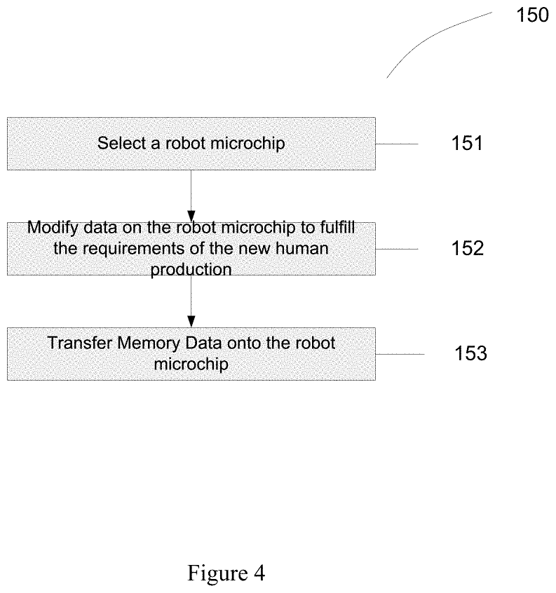

Step 150 in achieving human immortality is to integrate the memory data with an intelligent robot microchip, as illustrated in detailed steps shown in FIG. 4. Through integration, a new human-machine high-intelligence microchip is formed. Even though the current high-intelligence microchip for robots already possess a variety of advanced human functions such as hearing, vision, and smell, only the new human-machine high-intelligence microchip possesses the more advanced intelligence needed for immortality.

During integration, first select a robot microchip for the integration, shown as 151, then some of the original data in the robot microchip may need to be modified or deleted to fulfill the requirements of the new human production, this includes modifying some function commands to fit the need of the new human production, or deleting redundant commands if necessary, shown as 152. The retrieved and modified human memory data is then transferred to the robot microchip, shown as 153. After integration, a new human-machine high-intelligence microchip is formed. The human-machine high-intelligence microchip has the intelligence and functions of both immortal human brain microchip and the robot microchip, and is the key part to achieve the goal of human immortality. The immortal high-intelligence human-machine chip has the corresponding functions of the human brain, with hearing, vision, smell, and motor functions. It can act according to its own thinking and imagination. The changes in the outside world may generate stimulus in the human-machine microchip which in turn generate instinctive reflections and changes.

The immortal high-intelligence human-machine microchip is a self-learning chip, which can be self-improved and self-adjusted with the changes in the outside world, and it can better adapt to the changes and development of the world.

### Fabricate a Variety of High-Intelligence Human-Machine Bodies

Step 160 is to select a human-machine body for the person. It is necessary to produce a variety of high-intelligence man-machine bodies for the human-machine microchips to be implanted. The face of the human-machine bodies should be able to produce rich expression changes suitable for external changes. Using soft silica gel allows making subtle changes to the human face. The skin is almost the same as that of humans. There may have variety of man-machine bodies manufactured, such as different genders, height, age, beauty and ugliness. It is up to the person to choose a man-machine body to install the human-machine microchip.

The design of the human-machine body should model after the real human body, and the human-machine body should also have the same structure and material proportions like real human body, and the bones, joints, knuckles and other parts of the man-machine body are made of the highest quality alloy steel, which has formed the basic material for permanent survival.

The surface of the human-machine body is made of high-quality fine soft silica gel. It will have almost the same parts as the human body, such as eyes, armpit hair, human sweat glands, etc. The eyes can blink and the nose can breathe, etc.

Implant the Human-Machine High-Intelligence Microchip into the Human-Machine Body to Achieve Human Immortality

Step 160 is to implant the human-machine microchip into the selected human-machine body to create a new human.

Step 170 is to test the new human for normal functionalities.

At this stage, a variety of tests will be carried out:

1. Testing of personal cognition: This is to test of the new human of individual cognition, such as knowing your name, knowing your job title, experience, and personal history, etc.

2. Testing of personal emotion and characters. This is to test whether the attitudes, feelings, and emotions of the new human are the same as the original person when facing variety of things in the society.

3. Testing of personal mobility, that is, whether the new human has the same or even better mobility as the original person.

4. Testing of morality: This is to test whether the new human has the morality of a normal human being. Only those new humans who are fully qualified and have no criminal consciousness can be allowed to enter society.

5. Testing of recognition and experience of the new individual: this is to test whether new human recognizes he/she is a new individual, knows the similarities and differences between him/her and the original person.

After all these tests are conducted, and the new human may be sent into human society.

Important Characteristics of New Humans

This kind of high-intelligence new humans have many new characteristics.

1. Keep a healthy body;

2. No need to sleep and rest. The new human can work for a long time and to the maximum without fatigue.

3. The new human will not get sick. When the certain part of the body is worn to a considerable extent, just change the part.

4. Not afraid of general climate change, the new human may travel fora long time, even to travel to Mars or the Moon and live there.

Of course, the problems of male and female sexual intercourse and female reproduction in the new humans still need to be solved. This will be the research projects for the scientists and engineers. When these issues are resolved, the new humans may enjoy the same happy life as the current human beings.

The birth of the new human will produce extremely profound changes in the relationship between the new human and human beings. The relationship will become more friendly and united, because the mind of the new human has been revised during the integration stage, and its selfish and evil parts have been revised.

The body of the new human is more suitable for exploring, traveling and surviving on other planets. Therefore, the birth of the new human is the greatest innovation and practice in the history of human survival.

New humans may be imprisoned for a long time or have their human-machine microchips removed if they commit a crime, which is equivalent to sentenced to death.

### The Safeguard of Making New Humans

1. Safeguard in selecting a person to make a new human: The person who applies for new human production must have no criminal record.

2. Safeguard in social-economic status: Applicant for new human production must have a strong financial background, because the development and production process require a lot of money to complete.

3. Safeguard of new human social relations: especially the interaction between new humans and human beings needs to be carried out under intelligent supervision to prevent the possibility that the new humans are being manipulated and induced for deterioration of human nature.

4. Safeguard of new humans participating in normal social activities. New humans can participate in normal social activities and work, and enjoy the same rights and obligations as old humans to participate in social activities and work.

5. Enact laws for the new humans.

The terminology used herein is to describe particular exemplary embodiments only and is not intended to be limiting. As used herein, the singular forms “a,” “an,” and “the” may be intended to include the plural forms as well, unless the context expressly indicates otherwise. It will be further understood that the terms “comprise,” “comprises,” and/or “comprising,” “include,” “includes,” and/or “including,” when used in the present disclosure, specify the presence of stated features, steps, operation, elements, and/or components, but do not preclude the presence or addition of one or more other features, steps, operation, elements, components, and/or groups thereof.

These and other features, and characteristics of the present disclosure, as well as the methods of operation and functions of the related elements of structure and the combination of parts and economies of manufacture, may become more apparent upon consideration of the following description with reference to the accompanying drawings, all of which form a part of the present disclosure. It is to be expressly understood, however, that the drawings are for illustration and description only, and are not intended to limit the scope of the present disclosure. It is understood that the drawings are not to scale.

The flowcharts used in the present disclosure illustrate operation that systems implement according to some embodiments of the present disclosure. It is to be expressly understood, the operation of the flowcharts may be implemented not in order. Conversely, the operation may be implemented in inverted order, or simultaneously. Moreover, one or more other operations may be added to the flowcharts. One or more operations may be omitted from the flowcharts.

Although some preferred steps for achieving human immortality in accordance with the present invention have been described herein with respect to a limited number of embodiments, those skilled in the art will recognize that various substitutions and modifications may be made to the specific features described above without departing from the scope and spirit of the invention as recited in the appended claims.

What is claimed is:

1. A method for achieving human immortality for a person using intelligent microchips, comprising:
implanting a human brain microchip into the person's brain;
capturing personal memory data of the person in the human brain microchip;
retrieving the personal memory data from the human brain microchip;
integrating the personal memory data with a robot microchip to form a human-machine microchip;
selecting a human-machine body for implanting the human-machine microchip;
implanting the human-machine microchip in the selected human-machine body to form a new human; and
testing the new human for normal functionalities.

2. A method for achieving human immortality for a person using intelligent microchips according to claim 1 further comprising fabricating the human brain microchip.

3. A method for achieving human immortality for a person using intelligent microchips according to claim 1 further comprising manufacturing variety of human-machine bodies, wherein the variety of human-machine bodies vary in gender, height, age, beauty and ugliness.

4. A method for achieving human immortality for a person using intelligent microchips according to claim 1, wherein retrieving the personal memory data from the human brain microchip comprising:
a) removing the human brain microchip from the person's brain;
b) retrieving the personal memory data from the human brain microchip;
c) inspecting the personal memory data for completeness;
d) repairing the personal memory data if necessary; and
e) modifying the personal memory data to conform with public moral.

5. A method for achieving human immortality for a person using intelligent microchips according to claim 1, wherein the integrating the personal memory data with a robot microchip comprising:
a) selecting a robot microchip;
b) modifying data on the robot microchip to fulfill the requirements of new human production; and
c) transferring the personal memory data to the robot microchip.
